(19) United States
(12) Patent Application Publication
(10) Pub. No.: US 2021/0317165 A1
(43) Pub. Date: Oct. 14, 2021

(54) ZP3 FRAGMENTS IN IMMUNOTHERAPY OF OVARIAN CANCER

(71) Applicant: LABORATOIRE HRA-PHARMA, Chatillon (FR)

(72) Inventors: Michèle RESCHE-RIGON, Paris (FR); Charlotte DUVAL, Les Lilas (FR); Eric TARTOUR, Paris (FR); Marie ANSON, Montreuil (FR)

(21) Appl. No.: 17/272,750

(22) PCT Filed: Sep. 2, 2019
(86) PCT No.: PCT/EP2019/073357

§ 371 Date: Mar. 2, 2021

(30) Foreign Application Priority Data
Sep. 3, 2018 (EP) 18306159.7

(51) Int. Cl. C07K 7/06; C07K 14/74; A61K 39/00

(52) U.S. Cl. CPC C07K 7/06; A61K 2039/585; A61K 39/0005; C07K 14/70539

(57) ABSTRACT

The invention relates to zona pellucida protein (ZP3) fragments, and their use in immunotherapy of ovarian cancer.

The present invention relates to the prevention or treatment of ovarian cancer and metastases thereof. More specifically, the invention relates to immunogenic peptides, useful in vaccines and pharmaceutical compositions for prevention or treatment of ovarian cancer and metastases thereof.

### BACKGROUND OF THE INVENTION

Ovarian cancer is a frequent cancer in women, with a fatality rate which is rather high, relative to other cancers of the female reproductive organs.

The main therapeutic options for ovarian cancer are surgery, chemotherapy, and sometimes radiation therapy.

Surgery is the main treatment for all stages and types of ovarian cancer. Total hysterectomy (removal of the uterus) and unilateral or bilateral salpingo-oophorectomy (removal of one or both ovaries and corresponding Fallopian tube) is the most common surgery performed. Nearby lymph nodes, omentum and any other tissues that look abnormal at the time of surgery may also be removed.

Radiation therapy is not commonly used to treat ovarian cancer because it often involves many organs in the abdomen and radiation therapy needs to be aimed at a small area. It may be used after surgery if chemotherapy cannot be used because of older age or health problems. It may be used to treat small areas of cancer that have come back or spread and to control symptoms of advanced ovarian cancer.

International patent application WO2007/058536 discloses zona pellucida (ZP) glycoprotein as a potential candidate for immunotherapy for ovarian cancer.

However there is still a need to develop novel therapeutic strategies to improve current treatments of ovarian cancers.

### SUMMARY OF THE INVENTION

The present inventors now propose an immunotherapy of ovarian cancer with certain short fragments of human zona pellucida glycoprotein 3 (hZP3).

The invention provides peptides consisting of the following amino acid sequences: ALVYSTFLL (SEQ ID NO: 4); FTVDVFHFA (SEQ ID NO: 5); LRLMEENWNA (SEQ ID NO: 6); CLVDGLTDA (SEQ ID NO: 12); and NMIYITCHL (SEQ ID NO: 13).

Preferably, the invention provides a peptide consisting of the following amino acid sequence FTVDVFHFA of SEQ ID NO: 5.

The invention further provides a construct comprising a peptide of SEQ ID NO: 4, 5, 6, 12 or 13, preferably SEQ ID NO: 5, conjugated to a peptide comprising at least one class II MHC restricted epitope.

Preferably, the invention further provides a construct comprising a peptide of SEQ ID NO: 4, 5, 6, 12 or 13, preferably SEQ ID NO: 5, conjugated to a peptide comprising at least one class II MHC restricted epitope selected from the group consisting of YSTFLLHDPRPVGNL (SEQ ID NO: 32), PVGNLSIVRTNRAEIPIEC (SEQ ID NO: 33), RSPTFHLGDAAHLQA (SEQ ID NO: 34), VFHFANDSRNMIYIT (SEQ ID NO: 35) and NDSRNMIYITCHLKVTLA (SEQ ID NO: 36).

Another subject of the invention is a pharmaceutical composition comprising at least one of said peptides or constructs, in combination with one or more pharmaceutically acceptable excipients.

The peptide, construct or composition is useful in prevention or treatment of ovarian cancer, or metastases thereof, in a human patient.

### LEGEND TO THE FIGURES

FIG. 1 is a graph that represents the number of CD8+ T cells producing IFNγ when activated in vitro by antigen-presenting cell (APC) presenting hZP3 pool of peptides of SEQ ID NO: 1, 2, 4, 5 and 6 (pool A) or pool of peptides of SEQ ID NO: 10, 12, 13, 15 and 17 (pool B), from spleen cells of mice vaccinated with pool of peptides A or B and GM-CSF/CpG adjuvant,

FIG. 2 is a graph that represents the number of CD8+ T cells producing IFNγ when activated in vitro by APC presenting hZP3 peptides (each peptide separately) from spleen cells of mice vaccinated with pool of peptides (A+B) and GM-CSF/CpG adjuvant in mice.

FIG. 3 is a graph that represents the percent of CD8+ T cells expressing CD107a/b in vaccinated mice after specific in vitro ZP3 stimulation (with pool of peptides or OVCAR3 cells).

### DETAILED DESCRIPTION OF THE INVENTION

The inventors first selected 30 peptides between 9 and 10 amino acids, from the sequence of the human zona pellucida protein ZP3 (hZP3), corresponding to potential HLA-A2-restricted T cell epitopes. Among them, the inventors then selected five hZP3 peptides (SEQ ID NO: 4, SEQ ID NO: 5, SEQ ID NO: 6, SEQ ID NO: 12 and SEQ ID NO: 13) based on their capacity to induce a strong CD8+ T cell response (TCD8 response) after three immunizations in a transgenic HLA-A2 mouse model. This response was characterized as highly cytotoxic, even after re-stimulation with OVCAR3 cell line (a human ovarian carcinoma cell line).

On this basis, it is herein disclosed a method for preventing and/or treating ovarian cancer or metastases thereof in a woman by inducing a primary immune response to ZP3 peptides. The method comprises administering at least one peptide as defined herein, said peptide comprising a class I MHC-restricted native zona pellucida T cell epitope that is capable of eliciting a CD8+ T-cell mediated immune response in vivo.

### Definitions

The term “patient” as used herein refers to a human female, regardless of the age, in need of a treatment against ovarian cancer or metastases thereof. The patient may be a juvenile, a pre-menopausal or a post-menopausal woman.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of: halting the onset, reducing the risk of development, reducing the incidence, delaying the onset, reducing the development, as well as increasing the time to onset of symptoms of a particular disorder. In the context of the present invention, the prophylactic treatment more particularly refers to the prevention of recurrence of ovarian cancer, or prevention of apparition or recurrence of metastases.

The term “ZP3 glycoprotein” refers to one of the four distinct human ZP glycoprotein families consisting of ZP1, ZP2, ZP3 and ZP4, wherein ZP3 equals ZPC according to the nomenclature proposed by Harris et al. (1994) DNA seq. 96:829-834. More in particular, the term hZP3 as used herein refers to the glycoprotein sequence of SEQ ID NO: 31.

The term “class I MHC restricted epitope” refers to a peptide sequence recognized by CD8 T lymphocytes (also called CD8+ cells) in association with class I MHC molecules.

The term “class II MHC restricted epitope” refers to a peptide sequence recognized by antigen-presenting cell (APC) in association with class II MHC molecules.

The term “conjugated to” or “in conjugation” as used herein refers to a bond between at least two peptides, by means of a direct fusion, or through a linker.

The term “immune checkpoint inhibitor” refers to any compound inhibiting the function of an immune checkpoint and typically includes antibodies, polypeptides, peptides, nucleic acid molecules and small molecules. Preferred immune checkpoint inhibitors are antibodies. Examples of immune checkpoints include programmed cell death protein 1 (PD-1), PD-L1, cytotoxic T-lymphocyte-associated antigen 4 (CTLA-4), IDO, T-cell Ig and mucin domain 3 (TIM3), LAG3, T-cell immunoreceptor with Ig and immunoreceptor tyrosine-based inhibitory motif (ITIM) domains (TIGIT), BTLA, V-domain Ig suppressor of T-cell activation (VISTA), inducible T-cell COStimulator (ICOS), killer Ig-like receptors (KIRs), and CD39. In preferred embodiments, immune checkpoint inhibitors are anti-CTLA-4, anti-PD-1 or anti-PD-L1 antibodies.

Peptide Preparation:

Peptides described herein can be synthesized using standard synthetic methods known to those skilled in the art, for example chemical synthesis or genetic recombination. In a preferred embodiment, peptides are obtained by stepwise condensation of amino acid residues, either by condensation of a preformed fragment already containing an amino acid sequence in appropriate order, or by condensation of several fragments previously prepared, while protecting the amino acid functional groups except those involved in peptide bond during condensation. In particular, the peptides can be synthesized according to the method originally described by Merrifield.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonyl), Br—Z (2-bromobenzyloyycarbonyl), Bzl (benzyl), Fmoc (9-fluorenylmcthoxycarbonyl), Mbh (4, 4′-dimethoxydibenzhydryl), Mtr (4-methoxy-2, 3, 6-trimethylbenzenesulphonyl), Trt (trityl), Tos (tosyl), Z (benzyloxycarbonyl) and Clz-Bzl (2, 6-dichlorobenzyl) for the amino groups; NO2 (nitro) and Pmc (2,2, 5,7, 8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method. Alternatively, the peptide may be synthesized using recombinant techniques. In this case, a nucleic acid and/or a genetic construct comprising or consisting of a nucleotidic sequence encoding a peptide according to the invention, polynucleotides with nucleotidic sequences complementary to one of the above sequences and sequences hybridizing to said polynucleotides under stringent conditions.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

Thus, in another aspect, it is provided a host or host cell that expresses (or that under suitable circumstances is capable of expressing) a peptide of the invention; and/or that contains a polynucleotide or genetic construct that encodes the peptide of the invention.

The method of producing the peptide may optionally comprise the steps of purifying said peptide, chemically modifying said peptide, and/or formulating said peptide into a pharmaceutical composition.

Constructs

The hZP3 fragment peptides herein identified (of SEQ ID NO: 4, 5, 6, 12 or 13) comprise class I MHC restricted epitope(s).

In a particular embodiment, the invention provides a construct comprising at least one of said peptides, conjugated to a peptide comprising at least one class II MHC restricted epitope. Such peptides comprising at least one class II MHC restricted epitope typically consist of sequences of 6 to 25 amino acids, preferably 13 to 20 amino acids.

It is to be understood that such construct does not consist in, nor comprise, hZP3.

In one embodiment, said peptide comprising at least one class II MHC restricted epitope is a fragment of native ZP3. Such class II MHC restricted epitopes may be identified and selected as described in Example 5 below. Still preferably, said class II MHC restricted epitopes from hZP3 are chosen from the group consisting of YSTFLLHDPRPVGNL (SEQ ID NO: 32), PVGNLSIVRTNRAEIPIEC (SEQ ID NO: 33), RSPTFHLGDAAHLQA (SEQ ID NO: 34), VFHFANDSRNMIYIT (SEQ ID NO: 35) and NDSRNMIYITCHLKVTLA (SEQ ID NO: 36).

In another embodiment, said peptides comprising a class II MHC restricted epitope are not hZP3 fragment peptides, nor any fragment from a ZP3 from another species.

The hZP3 fragment of the invention can be conjugated to a peptide comprising at least one class II MHC restricted epitope, either directly, as a fusion protein, or through a linker.

The term “linker” as used herein refers to any peptide linker or cross-linking non-peptide linker. A peptide linker typically comprises 1 to 20 amino acids, preferably 4 to 15, still preferably 4 to 10 amino acids. Some preferred examples are Gly-Ser linkers such as tetraglycyl-seryl-triglycyl-serine peptide, or polyalanine linkers.

Alternatively, a non-peptide conjugation can involve the use of chemical groups that react with primary amines (—NH2) that exist at the N-terminus of each polypeptide chain and in the side-chain of lysine (Lys, K) amino acid residues. There are numerous synthetic chemical groups that will form chemical bonds with primary amines. These include isothiocyanates, isocyanates, acyl azides, NHS (N-hydroxysuccinimide) esters, sulfonyl chlorides, aldehydes, such as formaldehyde, glyoxals, epoxides, oxiranes, carbonates, aryl halides, imidoesters, carbodiimides, anhydrides, and fluorophenyl esters. Most of these conjugate to amines by either acylation or alkylation. NHS esters and imidoesters are the most preferred amine-specific functional groups that are incorporated into reagents for protein crosslinking Examples of cross-linking agents include dimethyl suberimidate (DMS), bissulfosuccinimidyl suberate (BS3), or disuccinimidyl suberate (DSS).

Further Protection Against Proteolysis or Improvement of Immunogenicity:

The N- and C-termini of the peptides or constructs described herein may be optionally protected against proteolysis. For instance, the N-terminus may be in the form of an acetyl group, and/or the C-terminus may be in the form of an amide group. Internal modifications of the peptides to be resistant to proteolysis are also envisioned, e.g. wherein at least a —CONH— peptide bond is modified and replaced by a (CH2NH) reduced bond, a (NHCO) retro-inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH-bond.

For instance the peptide may be modified by acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptides of the invention may be composed of amino acid(s) in D configuration, which render the peptides resistant to proteolysis.

To improve the immunogenicity, immuno-stimulating moieties may be attached, e.g. as in the constructs described above.

To enhance the solubility of the peptide, addition of charged or polar amino acids may be used, in order to enhance solubility and increase stability in vivo.

For immunization purposes the aforementioned immunogenic peptides according to the invention may also be fused with proteins such as but not limited to tetanus toxin/toxoid, diphtheria toxin/toxoid or other carrier molecules. The polypeptides may also be advantageously fused to heatshock proteins, such as recombinant endogenous (murine) gp96 (GRP94) as a carrier for immunodominant peptides as described in (Rapp and Kaufmann, Int Immunol. 2004, 16(4):597-605; Zugel, Infect Immun. 2001 June; 69(6):4164-7) or fusion proteins with Hsp70 (WO9954464).

In another aspect of the invention, peptides are covalently bound to a polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide half-life is increased by including the peptide in a biodegradable and biocompatible polymer material for drug delivery system forming microspheres. Polymers and copolymers are, for instance, poly(D,L-lactide-co-glycolide) (PLGA) (as illustrated in US2007/0184015, SoonKap Hahn et al).

Immunotherapy of the Ovarian Cancer:

It is herein described a method for preventing and/or treating ovarian cancer or metastases thereof in a woman in need thereof, by administering the woman with an effective amount of at least one of the peptides ALVYSTFLL (SEQ ID NO: 4); FTVDVFHFA (SEQ ID NO: 5); LRLMEENWNA (SEQ ID NO: 6); CLVDGLTDA (SEQ ID NO: 12); and NMIYITCHL (SEQ ID NO: 13), or a combination thereof.

In a particular embodiment, the method may be applied as adjunctive therapy during or following treatment of patients using any of the conventional methods, including, for example, oophorectomy, radiation therapy and/or chemotherapy.

The peptide(s) can be administered as the only active ingredient or in combination with another active agent, e.g. an anti-tumor agent such as chemotherapeutic agents, including immune checkpoint inhibitors, in particular anti-CTLA-4, anti-PD-1 and/or anti-PD-L1 antibodies, inhibitors of DNA replication such as DNA binding agents in particular alkylating or intercalating drugs, antimetabolite agents such as DNA polymerase inhibitors, or topoisomerase I or II inhibitors, or with anti-mitogenic agents such as alkaloids.

The peptides described herein may also useful for treating primary ovarian cancer or metastases thereof, as well as for preventing metastases and/or recurrence of ovarian cancer optionally after or in combination with other methods of treatment, such as described above.

The peptides are helpful in eradicating any persistent microscopic malignancy, and/or preventing or delaying relapses.

Pharmaceutical Compositions:

The peptides of the invention may be administered by any convenient route including parenteral, e.g. intravenous, transdermal, subcutaneous, mucosal, intramuscular, intrapulmonary, intranasal route or by oral, rectal, vaginal or topical route. Intra-tumoral administration is also contemplated. Parenteral, oral or vaginal routes are preferred.

The peptides are typically formulated in association with a pharmaceutically acceptable carrier. The pharmaceutical composition may also include any other active principle, such as in particular an anti-tumor agent, such as those described above.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product.

The present method may further comprise the administration, preferably the co-administration, of at least one adjuvant. Adjuvants may comprise any adjuvant known in the art of vaccination and may be selected using textbooks like Current Protocols in Immunology, Wiley Interscience, 2004.

Adjuvants are herein intended to include any substance or compound that, when used, in combination with an antigen, to immunise a human or an animal, stimulates the immune system, thereby provoking, enhancing or facilitating the immune response against the antigen, preferably without generating a specific immune response to the adjuvant itself. Preferred adjuvants enhance the immune response against a given antigen by at least a factor of 1.5, 2, 2.5, 5, 10 or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens.

A number of adjuvants are well known to one skilled in the art. Suitable adjuvants include e.g. Granulocyte-macrophage colony-stimulating factor (GM-CSF), cytosine-phosphate-guanine dinucleotide (CpG), incomplete Freund's adjuvant, alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxy-phosphoryloxy)-ethylamine (CGP 19835A, referred to as MTP-PE), DDA (2 dimethyldioctadecylammonium bromide), polylC, Poly-A-poly-U, RIBI™, GERBU™, Pam3™, Carbopol™, Specol™, Titermax™, tetanus toxoid, diphtheria toxoid, meningococcal outer membrane proteins, diphtheria protein CRM197. Preferred adjuvants comprise a ligand that is recognised by a Toll-like-receptor (TLR) present on antigen presenting cells. Various ligands recognised by TLR's are known in the art and include e.g. lipopeptides (see e.g. WO 04/110486), lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications.

The present method may further comprise the administration, preferably the co-administration, of a CD40 binding molecule in order to enhance a CTL response and thereby enhance the therapeutic effects of the methods and compositions of the invention. The use of CD40 binding molecules is described in WO 99/61065. The CD40 binding molecule is preferably an antibody or fragment thereof or a CD40 Ligand or a variant thereof, and may be added separately or may be comprised within a composition according to the current invention.

In another embodiment, immune checkpoint inhibitors, in particular anti-CTLA-4, anti-PD-1 and anti-PD-L1 antibodies, may be administered in combination with the peptides or constructs of the invention. Such inhibitors may be added separately or may be comprised within a composition according to the present invention.

For therapeutic applications, the present immunogenic polypeptides or nucleic acid sequences encoding them or the present compositions comprising these polypeptides or nucleic acid sequences encoding them are administered to a patient suffering from an ovarian tumour and possibly metastases thereof or to a patient that has received other methods of treating ovarian tumours, e.g. any of the conventional methods described herein before, in an amount sufficient to induce a primary autoimmune response directed against native ZP glycoproteins and tissue cells expressing ZP glyoproteins. An amount sufficient to accomplish this is defined as a “therapeutically-” or “prophylactically-effective dose”. Such effective dosages will depend on a variety of factors including the condition and general state of health of the patient. Thus dosage regimens can be determined and adjusted by trained medical personnel to provide the optimum therapeutic or prophylactic effect.

The dosing is selected by the skilled person so that an anti-cancerous effect is achieved, and depends on the route of administration and the dosage form that is used. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

In the present method the one or more peptides are typically administered at a dosage of about 1 μg/kg patient body weight or more at least once. Often dosages are greater than 10 μg/kg. The dosages preferably range from 1 μg/kg to 1 mg/kg. Preferably typical dosage regimens comprise administering a dosage of 1-1000 μg/kg, more preferably 10-500 μg/kg, still more preferably 10-150 μg/kg, once, twice or three times a week for a period of one, two, three, four or five weeks.

Another aspect of the disclosure comprises ex vivo administration of a composition comprising the present peptides to mononuclear cells from the patient blood, particularly dendritic cells (DC) isolated therefrom. A pharmaceutical to facilitate harvesting of DC can be used, such as Progenipoietin™ (Monsanto, St. Louis, Mo.) or GM-CSF/IL-4. After pulsing the DC with peptides and washing to remove unbound peptides, the DC are reinfused into the patient. In this aspect, a composition is provided comprising peptide-pulsed DC which present the pulsed peptide epitopes in HLA molecules on their surfaces. Methods of inducing an immune response employing ex vivo peptide-pulsed DC are well known to the skilled person.

The figures and examples illustrate the invention without limiting its scope.

### EXAMPLES

### Example 1: In Silico Identification of Peptides of Human Zona Pellucida Protein ZP3 Potentially Able to Induce a TCD8 Response in Human

The sequence of the human zona pellucida protein ZP3 (hZP3) was submitted to the in silico prediction software NETMHCPAN 3.0 prediction program” (http://www.cbs.dtu.dk/services/NetMHCpan/) in order to identify the potential HLA-A2-restricted T cell epitopes. Affinity of hZP3 peptides for HLA-A2 molecule was predicted. Sequences were retrieved from the NETMHCPAN 3.3 and ranked based on their score of binding, reported as percentile. Only peptides with a percentile below 2% were retained. 30 peptides (9 to 10 amino acids) were found below the positivity threshold of 2% and 14 of them were even below 1%. Some of these peptides are very overlapping corresponding to size variants of peptides but might correspond to different T cell epitopes.

TABLE 1
peptides of human ZP3 predicted to bind
HLA-A2 molecule

| In silico | | | SEQ ID | Affinity | |
|---|---|---|---|---|---|
| reference | Position | Peptide | NO: | (nM) | % Rank |
| 1 | 115 | FLLHDPRPV | SEQ ID | 5.1 | 0.04 |
| | | | NO: 1 | | |
| 2 | 175 | RLMEENWNA | SEQ ID | 5.2 | 0.04 |
| | | | NO: 2 | | |
| 3 | 11 | LLLWGSTEL | SEQ ID | 15.3 | 0.2 |
| | | | NO: 3 | | |
| 4 | 109 | ALVYSTFLL | SEQ ID | 21.3 | 0.3 |
| | | | NO: 4 | | |
| 5 | 263 | FTVDVFHFA | SEQ ID | 19.9 | 0.3 |
| | | | NO: 5 | | |
| 6 | 174 | LRLMEENWNA | SEQ ID | 28.6 | 0.4 |
| | | | NO: 6 | | |
| 7 | 388 | VLLGVGLAV | SEQ ID | 24.4 | 0.4 |
| | | | NO: 7 | | |
| 8 | 388 | VLLGVGLAVV | SEQ ID | 36.0 | 0.5 |
| | | | NO: 8 | | |
| 9 | 399 | SLTLTAVILV | SEQ ID | 32.7 | 0.5 |
| | | | NO: 9 | | |
| 10 | 367 | FLDRRGDHEV | SEQ ID | 53.2 | 0.7 |
| | | | NO: 10 | | |
| 11 | 389 | LLGVGLAVV | SEQ ID | 58.0 | 0.7 |
| | | | NO: 11 | | |
| 12 | 239 | CLVDGLTDA | SEQ ID | 79.1 | 0.9 |
| | | | NO: 12 | | |
| 13 | 276 | NMIYITCHL | SEQ ID | 76.6 | 0.9 |
| | | | NO: 13 | | |
| 14 | 387 | VVLLGVGLAV | SEQ ID | 90.9 | 1 |
| | | | NO: 14 | | |
| 15 | 114 | TFLLHDPRPV | SEQ ID | 101.9 | 1.1 |
| | | | NO: 15 | | |
| 16 | 401 | TLTAVILVL | SEQ ID | 113.6 | 1.1 |
| | | | NO: 16 | | |
| 17 | 64 | KLIRAADLTL | SEQ ID | 129.9 | 1.2 |
| | | | NO: 17 | | |
| 18 | 211 | RLFVDHCVAT | SEQ ID | 132.7 | 1.3 |
| | | | NO: 18 | | |
| 19 | 277 | MIYITCHLKV | SEQ ID | 133.8 | 1.3 |
| | | | NO: 19 | | |
| 20 | 175 | RLMEENWNAE | SEQ ID | 174.4 | 1.5 |
| | | | NO: 20 | | |
| 21 | 400 | LTLTAVILV | SEQ ID | 192.7 | 1.6 |
| | | | NO: 21 | | |
| 22 | 10 | CLLLWGSTEL | SEQ ID | 211.6 | 1.7 |
| | | | NO: 22 | | |
| 23 | 232 | TIVDFHGCLV | SEQ ID | 216.0 | 1.7 |
| | | | NO: 23 | | |
| 24 | 389 | LLGVGLAVVV | SEQ ID | 225.7 | 1.7 |
| | | | NO: 24 | | |
| 25 | 275 | RNMIYITCHL | SEQ ID | 241.8 | 1.8 |
| | | | NO: 25 | | |
| 26 | 400 | LTLTAVILVL | SEQ ID | 251.6 | 1.8 |
| | | | NO: 26 | | |
| 27 | 42 | VLVECQEATL | SEQ ID | 260.0 | 1.9 |
| | | | NO: 27 | | |
| 28 | 108 | DALVYSTFLL | SEQ ID | 269.3 | 1.9 |
| | | | NO: 28 | | |
| 29 | 397 | VVSLTLTAV | SEQ ID | 263.1 | 1.9 |
| | | | NO: 29 | | |
| 30 | 115 | FLLHDPRPVG | SEQ ID | 305.6 | 2 |
| | | | NO: 30 | | |

Among those 30 peptides, 10 have been selected according to their predicted affinity for HLA-A2 and their localization in the hZP3 sequence, for synthesis and further experiments in HLA-A2 transgenic mice.

TABLE 2
peptides of human ZP3 selected for synthesis
and in vivo experiments

| Reference | | |
|---|---|---|
| Peptide | | |
| number from | | |
| in silico | | |
| prediction | Sequence | SEQ ID |
| 1 | H-FLLHDPRPV-OH | SEQ ID NO: 1 |
| 2 | H-RLMEENWNA-OH | SEQ ID NO: 2 |
| 4 | H-ALVYSTFLL-OH | SEQ ID NO: 4 |
| 5 | H-FTVDVFHFA-OH | SEQ ID NO: 5 |
| 6 | H-LRLMEENWNA-OH | SEQ ID NO: 6 |
| 10 | H-FLDRRGDHEV-OH | SEQ ID NO: 10 |
| 12 | H-CLVDGLTDA-OH | SEQ ID NO: 12 |
| 13 | H-NMIYITCHL-OH | SEQ ID NO: 13 |
| 15 | H-TFLLHDPRPV-OH | SEQ ID NO: 15 |
| 17 | H-KLIRAADLTL-OH | SEQ ID NO: 17 |

### Example 2: In Vivo Characterization of Immune Response Induced by Peptides Administration in HLA-A2 Transgenic Mice Model

Immunogenicity of selected hZP3 MHC 1 peptides was assessed in HLA-A2/DR1 transgenic mice model.

HLA-A2/DR1 transgenic mice received three subcutaneous administrations of two different peptide pools and GM-CSF (Granulocyte Macrophage Colony Stimulating Factor)/CpG adjuvant as follows:

A first group (group 1) of mice received 100 μg/mice of a pool of peptides SEQ ID NO: 1, 2, 4, 5 and 6 (pool A) and GM-CSF adjuvant at Day 0 (D0). At D1, CpG wad injected. 100 μg/mice of peptides of pool A was injected at D14 and D30. Mice were sacrificed and analyzed at D40.

A second group (group 2) of mice was treated using the same protocol and same concentrations, but replacing the pool A of peptides by a pool of peptides SEQ ID NO: 10, 12, 13, 15 and 17 (pool B).

A third group (group 3) of mice consisted of naïve control mice.

TABLE 3
protocol for pools of peptides A and B subcutaneous
administrations to HLA-A2/DR1 transgenic mice.

| | Group 1 | Group 2 | Group 3 |
|---|---|---|---|
| D0 | Pool A (100 μg/mice) + | pool B (100 μg/mice) + | Naive |
| | GM-CSF | GM-CSF | |
| D1 | CpG | CpG | |
| D14 | Pool A (100 μg/mice) | pool B (100 μg/mice) | |
| D30 | pool A (100 μg/mice) | pool B (100 μg/mice) | |

| | |
|---|---|
| D40 | Sacrifice and analysis |

Specific antibody response was assessed with ELISA assay, by measuring anti-ZP3 IgG response, using sera of mice. No antibody response was found (no antibody specific to ZP3 was detected).

The ZP3-specific TCD4 and TCD8 response was measured with ex vivo stimulation of TCD4 or TCD8 from spleen of mice, using antigen-presenting cell (APC) pulsed with peptides (pool A or pool B). The number of activated TCD4 or TCD8 cells was measured by ELISPOT IFNγ. No ZP3 specific TCD4 response was found but a strong ZP3 specific TCD8 response for the two pools of peptides was measured (FIG. 1).

The histopathology of ovaries was performed to assess potential necrosis in ovarian tissue, by hematoxylin and eosin staining (H&E staining). No sign of necrosis in ovaries of immunized mice was observed. Ovaries appear normal.

### Example 3: In Vivo Characterization of TCD8 Response Induced by Peptides Administration in HLA-A2 Transgenic Mice Model

TCD8 response induced by hZP3 MHC1 peptides was further characterized and dominant peptides were identified in HLA-A2/DR1 mice model.

HLA-A2/DR1 transgenic mice received three subcutaneous administrations of peptides pool and GM-CSF/CpG adjuvant as follow:

A first group (group 1) of mice received 100 μg/mice of a pool of peptides A+B (SEQ ID NO: 1, 2, 4, 5, 6, 10, 12, 13, 15 and 17) and GM-CSF adjuvant at Day 0 (D0). At D1, CpG wad injected. 100 μg/mice of pool of peptides A+B was injected at D14 and D30. Mice were sacrificed and analyzed at D40.

A second group (group 2) of mice consisted of naïve control mice.

TABLE 4
protocol for pool of peptides A + B subcutaneous
administrations to HLA-A2/DR1 transgenic mice

| | Group 1 | Group 2 |
|---|---|---|
| D0 | pool of peptides A + B (100 μg/mice) + | Naive |
| | GM-CSF | |
| D1 | CpG | |
| D14 | pool of peptides A + B (100 μg/mice) | |
| D30 | pool peptides A + B (100 μg/mice) | |

| | |
|---|---|
| D40 | Sacrifice and analysis |

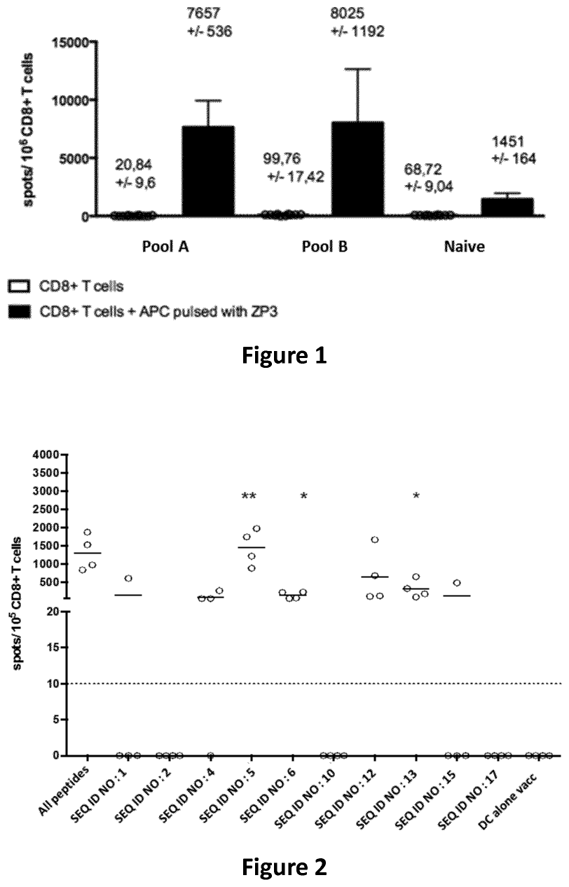

The ZP3-specific TCD8 response was measured by ex vivo stimulation of TCD8 from spleen of mice with APC pulsed with peptides (each peptides separately). The number of activated TCD8 cells was measured by ELISPOT IFNγ. Results led to the identification of three ZP3 peptides (peptides of SEQ ID NO: 5, 6 and 13) inducing a strong specific TCD8 response (statistically significant). Two other ZP3 peptides (SEQ ID NO: 4 and 12) induced smaller response but was not significant, due to high variation (FIG. 2).

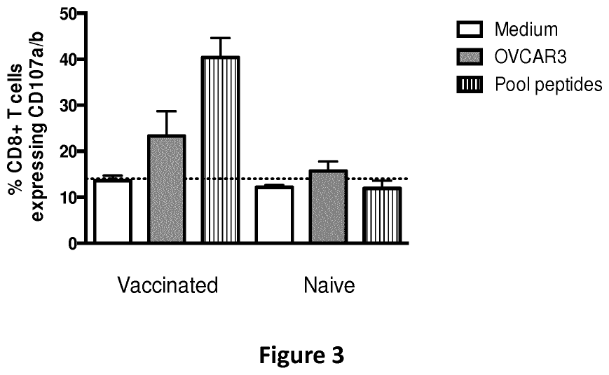

The cytotoxicity of TCD8 was observed using a degranulation assay. TCD8 from spleen of mice were stimulated ex vivo with APC pulsed with peptides (pool of peptides A+B) or with OVCAR3 cells. Staining of CD107a/b was performed before analysis by flow cytometry to assess degranulation of TCD8. A significant increase of percent of cytotoxic TCD8 (TCD8 expressing CD107a/b) in response to hZP3 peptides stimulation was observed in mice immunized with hZP3 peptides (FIG. 3). To a lesser extent, a significant increase of cytotoxic TCD8 was also present in response to OVCAR3 cells stimulation (human ovarian tumor cell line expressing ZP3).

### CONCLUSION

30 peptides of the hZP3 protein with a potential strong affinity for HLA-A2 molecule were identified, based on in silico approach. Among them, five hZP3 peptides (SEQ ID NO: 4, 5, 6, 12 and 13), able to induce a strong TCD8 response after three immunizations in a transgenic HLA-A2 mouse model were selected. Moreover, this response was characterized as highly cytotoxic even after re-stimulation with OVCAR3 cell line.

### Example 4: In Silico Immunogenicity Prediction of Potential HLA-DR-Restricted T Cell Epitopes of Human Zona Pellucida Protein ZP3

The sequence of the human ZP3 was submitted to the in silico NetMHCPAN3.1 prediction software (http://tools.iedb.org/mhciii) in order to identify the potential HLA-DR-restricted T cell epitopes. The ZP3 sequence was submitted for each of the alleles HLA-DRB1*01:01, 03:01, 04:01, 07:01, 08:02, 09:01, 11:01, 12:01, 13:01, 15:01, HLA-DRB3*01:01, HLA-DRB3*02:02, HLA-DRB4*01:01, HLA-DRB5*01:01, which are the most preponderant allotypes in the worldwide population.

Peptide cores of ZP3 potential CD4 T cell epitopes were retrieved from the NETMHCPAN 3.1 for the selected alleles and ranked on the basis of the number of bound alleles. Only core peptides with a percentile below 10% were retained. Ten core peptides bound to at least 3 different HLA-DR allotypes and were selected. These core regions were localized in the ZP3 sequences to identify their flanking regions. 10 peptides were defined, and 5 were eventually selected, based on their localization in the hZP3 sequence (Table 5):

TABLE 5
selected class II MHC peptides

| Amino acid position | Sequence | SEQ IN NO |
|---|---|---|
| 112-126 | YSTFLLHDPRPVGNL | 32 |
| 122-140 | PVGNLSIVRTNRAEIPIEC | 33 |
| 186-200 | RSPTFHLGDAAHLQA | 34 |
| 267-281 | VFHFANDSRNMIYIT | 35 |
| 272-419 | NDSRNMIYITCHLKVTLA | 36 |

1. A hZP3 fragment peptide selected from the group consisting of
(SEQ ID NO: 4)
ALVYSTFLL;
(SEQ ID NO: 5)
FTVDVFHFA;
(SEQ ID NO: 6)
LRLMEENWNA;
(SEQ ID NO: 12)
CLVDGLTDA;
and
(SEQ ID NO: 13)
NMIYITCHL.

2. The hZP3 fragment peptide according to claim 1, wherein said peptide is the peptide of SEQ ID NO: 5.

3. A construct comprising at least one peptide as defined in claim 1 or 2, conjugated to a peptide comprising at least one class II MHC restricted epitope.

4. The construct as defined in claim 3, wherein the peptide comprising at least one class II MHC restricted epitope is selected from the group consisting of YSTFLLHDPRPVGNL (SEQ ID NO:32), PVGNLSIVRTNRAEIPIEC (SEQ ID NO:33), RSPTFHLGDAAHLQA (SEQ ID NO: 34), VFHFANDSRNMIYIT (SEQ ID NO: 35) and NDSRNMIYITCHLKVTLA (SEQ ID NO: 36).

5. A pharmaceutical composition comprising at least one peptide as defined in claim 1 or 2, or at least one construct as defined in claim 3 or 4, in combination with one or more pharmaceutically acceptable excipients.

6. The composition of claim 5, further comprising an adjuvant.

7. The hZP3 fragment peptide as defined in claim 1 or 2, the construct as defined in claim 3 or 4, or the composition of claim 5 or 6, for use in prevention or treatment of ovarian cancer in a human patient.

8. The hZP3 fragment peptide as defined in claim 1 or 2, the construct as defined in claim 3 or 4, or the composition of claim 5 or 6, for use according to claim 7, in prevention or treatment of metastases of ovarian cancer and/or recurrence of ovarian cancer in a human patient.

9. The hZP3 fragment peptide as defined in claim 1 or 2, the construct as defined in claim 3 or 4, or the composition of claim 5 or 6, for use according to claim 7 or 8, wherein the peptide or composition is to be administered by parenteral or vaginal route.

10. The hZP3 fragment peptide as defined in claim 1 or 2, the construct as defined in claim 3 or 4, or the composition of claim 5 or 6, for use according to any of claims 7 to 9, wherein the peptide or composition is to be co-administered with at least one adjuvant.

11. The hZP3 fragment peptide as defined in claim 1 or 2, the construct as defined in claim 3 or 4, or the composition of claim 5 or 6, for use according to any of claims 7 to 10, wherein the peptide or composition is to be administered as an adjunct therapy to oophorectomy, radiation therapy and/or chemotherapy.


SEQUENCE LISTING (36 sequences)

SEQ ID NO: 1 - protein, 9 aa, Artificial Sequence
  Peptide of human ZP3
FLLHDPRPV

SEQ ID NO: 2 - protein, 9 aa, Artificial Sequence
  Peptide of human ZP3
RLMEENWNA

SEQ ID NO: 3 - protein, 9 aa, Artificial Sequence
  Peptide of human ZP3
LLLWGSTEL

SEQ ID NO: 4 - protein, 9 aa, Artificial Sequence
  Peptide of human ZP3
ALVYSTFLL

SEQ ID NO: 5 - protein, 9 aa, Artificial Sequence
  Peptide of human ZP3
FTVDVFHFA

SEQ ID NO: 6 - protein, 10 aa, Artificial Sequence
  Peptide of human ZP3
LRLMEENWNA

SEQ ID NO: 7 - protein, 9 aa, Artificial Sequence
  Peptide of human ZP3
VLLGVGLAV

SEQ ID NO: 8 - protein, 10 aa, Artificial Sequence
  Peptide of human ZP3
VLLGVGLAVV

SEQ ID NO: 9 - protein, 10 aa, Artificial Sequence
  Peptide of human ZP3
SLTLTAVILV

SEQ ID NO: 10 - protein, 10 aa, Artificial Sequence
  Peptide of human ZP3
FLDRRGDHEV

SEQ ID NO: 11 - protein, 9 aa, Artificial Sequence
  Peptide of human ZP3
LLGVGLAVV

SEQ ID NO: 12 - protein, 9 aa, Artificial Sequence
  Peptide of human ZP3
CLVDGLTDA

SEQ ID NO: 13 - protein, 9 aa, Artificial Sequence
  Peptide of human ZP3
NMIYITCHL

SEQ ID NO: 14 - protein, 10 aa, Artificial Sequence
  Peptide of human ZP3
VVLLGVGLAV

SEQ ID NO: 15 - protein, 10 aa, Artificial Sequence
  Peptide of human ZP3
TFLLHDPRPV

SEQ ID NO: 16 - protein, 9 aa, Artificial Sequence
  Peptide of human ZP3
TLTAVILVL

SEQ ID NO: 17 - protein, 10 aa, Artificial Sequence
  Peptide of human ZP3
KLIRAADLTL

SEQ ID NO: 18 - protein, 10 aa, Artificial Sequence
  Peptide of human ZP3
RLFVDHCVAT

SEQ ID NO: 19 - protein, 10 aa, Artificial Sequence
  Peptide of human ZP3
MIYITCHLKV

SEQ ID NO: 20 - protein, 10 aa, Artificial Sequence
  Peptide of human ZP3
RLMEENWNAE

SEQ ID NO: 21 - protein, 9 aa, Artificial Sequence
  Peptide of human ZP3
LTLTAVILV

SEQ ID NO: 22 - protein, 10 aa, Artificial Sequence
  Peptide of human ZP3
CLLLWGSTEL

SEQ ID NO: 23 - protein, 10 aa, Artificial Sequence
  Peptide of human ZP3
TIVDFHGCLV

SEQ ID NO: 24 - protein, 10 aa, Artificial Sequence
  Peptide of human ZP3
LLGVGLAVVV

SEQ ID NO: 25 - protein, 10 aa, Artificial Sequence
  Peptide of human ZP3
RNMIYITCHL

SEQ ID NO: 26 - protein, 10 aa, Artificial Sequence
  Peptide of human ZP3
LTLTAVILVL

SEQ ID NO: 27 - protein, 10 aa, Artificial Sequence
  Peptide of human ZP3
VLVECQEATL

SEQ ID NO: 28 - protein, 10 aa, Artificial Sequence
  Peptide of human ZP3
DALVYSTFLL

SEQ ID NO: 29 - protein, 9 aa, Artificial Sequence
  Peptide of human ZP3
VVSLTLTAV

SEQ ID NO: 30 - protein, 10 aa, Artificial Sequence
  Peptide of human ZP3
FLLHDPRPVG

SEQ ID NO: 31 - protein, 424 aa, Homo sapiens [sequence omitted]

SEQ ID NO: 32 - protein, 15 aa, Artificial Sequence
  Peptide of human ZP3
YSTFLLHDPRPVGNL

SEQ ID NO: 33 - protein, 19 aa, Artificial Sequence
  Peptide of human ZP3
PVGNLSIVRTNRAEIPIEC

SEQ ID NO: 34 - protein, 15 aa, Artificial Sequence
  Peptide of human ZP3
RSPTFHLGDAAHLQA

SEQ ID NO: 35 - protein, 15 aa, Artificial Sequence
  Peptide of human ZP3
VFHFANDSRNMIYIT

SEQ ID NO: 36 - protein, 18 aa, Artificial Sequence
  Peptide of human ZP3
NDSRNMIYITCHLKVTLA